(19) United States
(12) Patent Application Publication
(10) Pub. No.: US 2024/0402109 A1
(43) Pub. Date: Dec. 5, 2024

(54) Theramodynamic Source Monitoring System for Determining the Biodegradation Rate in Areas Contaminated by Petroleum Hydrocarbons and Computer-Implemented Method for Processing Monitored Raw Data

(71) Applicants: PETRÓLEO BRASILEIRO S.A. - PETROBRAS, Rio de Janeiro (BR); UNIVERSIDADE FEDERAL DE SANTA CATARINA - UFSC, Florianópolis (BR)

(72) Inventors: Marcus Paulus Martins Baessa, Rio de Janiero (BR); David Ciro Taborda, Florianópolis (BR); Adriana Ururahy Soriano, Rio de Janeiro (BR); Marcio Roberto Schneider, Florianópolis (BR); Admir José Giachini, Florianópolis (BR)

(21) Appl. No.: 18/666,456

(22) Filed: May 16, 2024

(30) Foreign Application Priority Data
May 29, 2023 (BR) 10 2023 0103766

(51) Int. Cl. G01N 25/48; G01K 3/10; G01N 33/24

(52) U.S. Cl. CPC G01N 25/4866; G01K 3/10; G01N 33/241

(57) ABSTRACT

The present invention refers to a thermodynamic system and a method to quantify the amount of oxidized methane and estimate the biodegradation rate of petroleum hydrocarbons in the contamination source zone in real time. The biodegradation rate is used to estimate the time required to reach safe levels in the concentration of contaminants in the soil. The system comprises a set of heat flux transducers distributed in the soil, different planes, a cylinder and a station with energy autonomy. The method includes analyzing the raw data, eliminating outliers caused by eventual probing failures, calculating the temperatures and their gradients for each moment measured at each observation point, calculating the temperature interpolant parameters for each moment using the temperatures and point derivatives, calculating the internal energy of the soil and the heat fluxes for each moment, calculating the cumulative value of energy transmitted, using a mathematical model to adjust the parameters of the decay curve, and identifying if there is any temporal dependence on the decay rates.

### RELATED APPLICATIONS

This application claims the benefit of Brazilian Patent Application No. BR 10 2023 0103766, filed May 29, 2023, the entire contents of which are explicitly incorporated by reference herein.

### FIELD

The present invention relates to the technical field of oil and gas; more specifically, it is related to monitoring the decay of contaminants in areas of oil, derivatives, and biofuel spills.

### BACKGROUND

It is presently known that accidental leaks in refining, transportation, and distribution operations of oil and derivatives result in contamination of soil and water resources. In addition to the socio-environmental consequences, the difficulty of extracting these contaminants from the environments impacted by remediation techniques based on hydraulic, pneumatic and chemical processes results in remediation costs that can reach hundreds of millions of reais per year for oil companies. Science on mitigating environmental impacts has shown that petroleum hydrocarbons are biodegradable and concentrations of their most toxic components can be reduced naturally over time. However, long-term analytical monitoring to demonstrate the efficiency of biodegradation is also costly, requiring complex logistics in remote areas.

In the above context, the present invention refers to a thermodynamic system and method for quantifying the natural biodegradation of free-phase petroleum hydrocarbons as a function of real-time monitoring of temperature of the affected area. From a vertical network of a simple set of temperature sensors at the source of contamination, the thermodynamic model converts the heat flux generated by biodegradation into hydrocarbon depletion rate, demonstrating in real time the decay of toxic substances. This novel alternative has high potential for reducing avoidable costs in the process of recovering contaminated areas, avoiding remediation in low-risk areas.

Thus, the present invention aims to use a system and a method as a basis for an instrumentation project that aims at the acquisition of field parameters for real-time monitoring of the biodegradation rate in areas contaminated by oil and derivatives, as an effective and less costly technological alternative in the long term, including in remote areas.

Consequently, in a terrestrial spill scenario, in the vast majority of cases, emergency action removes only part of the product (free phase), leaving a residual amount of hydrocarbons (residual phase) that cannot be removed due to technical and/or financial limitations. However, hydrocarbons can be degraded by native microorganisms, releasing methane, which diffuses into the soil towards the surface. In the process of methane diffusion, it undergoes oxidation, through an exothermic process that, therefore, releases energy in the form of heat. The heat produced is transmitted to the materials of the porous medium that make up the soil (grains, water and voids). Heat transfer follows a diffusive process whose speed is controlled by the thermal conductivity of the medium and its heat capacity. By knowing the thermal properties of the porous medium and measuring the fluctuations in temperature and heat flux in the subsurface, it is possible to solve the heat equation to measure the intensity of the heat source. This information is used to quantify the amount of oxidized methane and finally estimate the amount of contaminant degraded over time, i.e. the biodegradation rate of petroleum hydrocarbons in the contamination source zone. This process of biological degradation in the contamination source zone is one of the main factors that cause the decay of contaminants in the source zone, also known as Natural Source Zone Depletion (NSZD). The biodegradation rate can then be used to estimate the time required to reach safe levels in the concentration of contaminants in the soil.

In this sense, observing the limitations of the state of the art, it can be seen that the traditional approach for the remediation of contamination sources consists of the use of techniques such as: extraction by hydraulic and pneumatic methods, excavation and final disposal, heat or chemical oxidation, injection of vapors or remediating products (surfactants, oxidants and inputs for biodegradation). Depending on the complexity of the contamination scenario, it is noted that even combined techniques cannot solve the problem in the short term. In the case of using the NSZD technique, the application costs are associated with traditional monitoring, by chemical analysis methods or measurement of gas flows periodically (months), in the long term (years or decades).

Thus, the use of the system and method of the present invention proved to be a more promising way to demonstrate the occurrence of the natural biodegradation process involved in the NSZD technique, which consists of monitoring the heat flux at the source of contamination (in the soil), as an indirect measure of the decay of the mass of contaminants over time.

In view of this, and in order to solve the technical problems described above, with this invention, it is possible to observe environmental advantages such as the continuous monitoring of biodegradation processes in situ and, consequently, the biodegradation rate of contamination sources, the reduction of environmental monitoring costs and the overall cost of remediation, resulting in the reduction of operational expenditures (OPEX) during the recovery of environmental liabilities, promoting the application of technical and financial resources in areas that really represent greater risk, assisting in the management and control of avoidable costs, as well as a reduction in interventions in the contaminated area and carbon emissions compared to active remediation processes.

### STATE OF THE ART

In the state of the art, there are systems and methods to monitor subsurface conditions by estimating the biodegradation rate of contaminants in the soil; however, the systems and methods of the state of the art present drawbacks when compared to the thermodynamic system and the method of the present invention, in view that, in general, no method of the art allows to include the stratigraphy of the soil in the parameters of water retention and porosity of each layer of the soil, allowing to estimate the water content as a function of depth, which is used to correct the heat capacity and thermal conductivity of the soil at each depth to make a more accurate estimate of the internal energy of the monitored soil column and the heat fluxes in the control volume. In addition, the system of the present invention is based on a series of probes, which were developed to simultaneously measure the temperature and its three-dimensional gradient at each observation point, equipped with the estimation of the thermal conductivity of the soil at each depth, making it possible to estimate the three-dimensional heat flux at each point, which allows examining the validity of the model used.

Patent document US2020363359A, for example, refers to a system for monitoring subsurface conditions comprising: a first thermal sensor communicating with a data logger, the first thermal sensor transmitting to the data logger, at least one temperature obtained at a first subsurface location; a second thermal sensor communicating with the data logger, the second thermal sensor transmitting to the data logger, at least one temperature obtained at a second subsurface location; and a computing device in communication with the data logger, the computing device comprising at least one hardware processor and at least one memory for storing executable instructions that, when executed by at least one processor, are configured to: estimate a planar location of a subsurface heating or cooling source produced by an endothermic reaction or an exothermic reaction of organic material within a subsurface formation; calculate, using at least one temperature obtained at the first subsurface location and at least one temperature obtained at the second subsurface location, a first thermal parameter corresponding to an estimated rate of thermal change from a subsurface heating or cooling source and a second thermal parameter corresponding to an estimated rate of thermal change from the subsurface heating or cooling source based on the estimated location; convert the first thermal parameter and the second thermal parameter into a change rate of an amount of the organic material within the subsurface formation; and perform a location correction for remediation of subsurface formation based on the change rate of the amount of organic material within the subsurface formation.

However, the present invention calculates the parameters of the temperature interpolant for each moment using the temperatures and point derivatives differing from the document US2020363359A, as it stipulates two thermal parameters, a first for the surface and a second for the subsurface. Furthermore, contrary to the US2020363359A document, the present invention uses a set of transducers to determine the temperature and its three-dimensional gradient at the center of the transducer, the number and distribution of temperature transducers and the spacing between them depending on the geometry of the source of contamination in the subsurface of the area of interest.

In turn, document US2017023539A relates to the contamination of underground environments by oil and other light non-aqueous phase liquids (LNAPL), which is a widespread issue raising concerns about the transport of contaminants and the risks of water table pollution. Specifically, document US2017023539A includes the integration of the mathematical model along with a groundwater and heat transport model.

However, the present invention differs from document US2017023539A in that it uses a system of temperature transducers, in which the spacing between them depends on the geometry of the source of contamination in the subsurface of the area of interest, specifically of two configurations: 1D configuration consisting of a set of probes distributed vertically between the soil surface and the water table; and the 2D configuration employing a set of probes arranged horizontally at a depth of 0.5 m in the affected zone and a single probe in an unaffected zone with a location and stratigraphy similar to the affected one.

On the other hand, document US2021356450A protects methods and systems to stimulate and detect the biological degradation of hydrocarbons and biogeochemical cycles in contaminated soils. A method for long-term, in situ biogeochemical monitoring of subterranean soil carbon at a site, comprising monitoring over an extended period the levels of carbon-containing compounds in the subterranean soil, using one or more depletion-sensing wells that are arranged within the subterranean soil below a soil surface at the location and that remain in the subterranean soil for the extended period, each of the one or more depletion sensor wells having a plurality of sensors located in one or more isolated depth zones along the depletion sensor well. It is a subterranean soil monitoring system, characterized by comprising a network of depletion sensor wells that penetrate the soil surface, with each of the depletion sensor wells containing a depletion sensor.

The present invention differs from US2021356450A document, in view that the technology used in US2021356450A aims to stimulate and detect the biological degradation of hydrocarbons and biogeochemical cycles in contaminated soils, not allowing to include soil stratigraphy in the parameters of water retention and porosity of each soil layer as occurs in the present invention.

### BRIEF DESCRIPTION

The present invention relates to a thermodynamic system for real-time monitoring of contamination sources with application in NSZD (Natural Source Zone Depletion), comprising a set of heat flux transducers distributed in the soil, wherein said transducers monitor the heat generated by the biodegradation of petroleum hydrocarbons; in each plane, there is a pair of temperature probes, one with measurement points at angles 0° and 180° and the other with points at angles 90° and 270°, the cylinder diameter ranging between 3 and 10 cm depending on the quantity of probes to be attached to the rod, wherein the interior of the cylinder accommodates the installation wiring; the set of transducers determines the temperature and its three-dimensional gradient at the center of the transducer; each flux transducer comprises four temperature transducers arranged on the surface of a threaded cylindrical module in two planes perpendicular to the cylinder axis; the quantity and distribution of temperature transducers and the spacing between them depend on the geometry of the contamination source in the subsurface of the area of interest, specifically two configurations: 1D configuration, consisting of a set of probes vertically distributed between the soil surface and the water table; and 2D configuration employing a set of probes arranged horizontally at 0.5 m depth in the affected zone and a single probe in an unaffected zone with similar location and stratigraphy to the affected zone; additionally, atmospheric pressure and ambient temperature data, relative air and soil humidity, water table level in the monitored region, rainfall, and solar irradiance are monitored via commercial transducers coupled to the thermodynamic source monitoring system; the system also includes a station with energy autonomy (3) through solar panels and internal battery that transfers data via radio frequency, GPRS, or 3G/4G/5G; wherein the system acquires processed information to estimate the depletion of contaminant mass through a processing station: selecting the 1D or 2D configuration; identifying the product spilled into the soil; collecting soil samples at different depths to determine porosity in laboratory, for obtaining retention parameters, conductivity, and heat capacity at different depths; determining probe characteristics by measuring the gain and offset of each at the time of installation; recording the GPS position of the station and the vertical and horizontal positions of the probes relative to the station rod; and entering the characteristics of the spilled substance, soil, probes, and assembly into a database that will receive the measurements collected by the station.

The invention also comprises a computer-implemented method for processing raw data monitored by the system in real time, which performs the steps of: analyzing the raw data; eliminate outliers caused by eventual probing failures; calculate the temperatures and their gradients for each moment measured at each observation point using the geometry of the probes, calculate the temperature interpolant parameters for each moment using the point temperatures and derivatives, calculate the internal energy of the soil and the heat fluxes for each moment, set 1D configuration or 2D configuration; calculate the cumulative value of energy transmitted through the plane over time for 2D configurations or of energy produced per unit area for 1D configurations, where the accumulated energy over time is an indirect measure of the amount of methane that has been oxidized in the vadose zone, use an exponential model to adjust the parameters of the decay curve and identify if there is any temporal dependence on decay rates as a result of seasonal temperature or water table level, perform a projection of depletion over time, based on the modeling of the decay rate, and define a function that calculates the time needed to reach some user-defined concentration level.

### BRIEF DESCRIPTION OF THE DRAWINGS

To enhance this description and provide a clearer insight into the features of the present invention, a set of accompanying figures is included. These figures exemplify, though not exhaustively, the preferred embodiment.

FIG. 1 shows a general scheme of the invention system, according to a preferred embodiment of the present invention. Also in FIG. 1, the following constituent elements stand out: heat flux transducers distributed on the soil (1), station with energy autonomy (2), processing station (3) and user interface (4).

FIG. 2 is a general diagram of the way in which the steps of the method are communicated, according to a preferred embodiment of the present invention.

FIG. 3 depicts a prototype thermodynamic system for monitoring sources installed in the field, according to a preferred embodiment of the present invention.

FIG. 4 is a perspective view of the prototype installed in the field, according to a preferred embodiment of the present invention.

### DETAILED DESCRIPTION

The present invention refers to a system comprising a set of heat flux transducers (1), which are distributed in the soil to monitor the heat generated by the biodegradation of petroleum hydrocarbons and, consequently, biodegradation. This system also has sensors for temperature, moisture, water level, solar radiation, precipitation (and others if necessary for other purposes), energy source (battery and solar panels), electronic controllers, multiplexers and wireless communication devices (RF, 3G, 4G or 5G).

The set of heat flux transducers (1) determines the temperature and its three-dimensional gradient at the center of the transducer. Each flux transducer consists of four temperature transducers arranged on the surface of a threaded cylindrical module in two planes perpendicular to the cylinder axis. In each plane there is a pair of temperature probes, one with measurement points at the 0° and 180° angles and the other with points at the 90° and 270° angles. The cylinder diameter is between 3 and 10 cm depending on the number of probes that will be attached to the rod, as the inside of the cylinder accommodates the installation wiring. Similarly, the distance between the planes of the probes is between 3 and 10 cm.

The number and distribution of temperature transducers and the spacing between them are customized for each contamination scenario in the area of interest, depending on the geometry of the source of contamination in the subsurface of the area of interest, comprising an embodiment of the invention with a 1D configuration or with a 2D configuration.

Specifically, a preferred embodiment of the invention with a 1D configuration, consists of a set of probes distributed vertically between the soil surface and the water table. In general, the first probe is placed at a depth of 0.2 m and a uniform spacing between probes is applied until it reaches the water table, respecting that there are no less than 5 observation points and that the spacing between probes is not greater than 1.0 m. For example, if the water table is only 1.0 m deep, the criterion of the minimum number of probes (5 probes) is used, which results in a spacing of 0.2 m between probes. However, if the water table is located at a depth of 10.0 m, the criterion of maximum spacing between probes (10 probes or more), evenly spaced every 1.0 m or less, is used. The quality of the temperature interpolant function is not severely affected by the spacing between observation points, since the probe provides the directional derivatives of the temperature field, which allows to define the interpolant voltage at each point, which would not be possible by measuring the temperature at each point alone. The vertical configuration provides a detailed description of the temperature profile in the affected region, which can be useful for estimating the methane production and oxidation zones in the soil column.

In another preferred embodiment of the invention, there is a 2D configuration that employs a set of probes arranged horizontally at a depth of 0.5 m in the affected zone and a single probe in an unaffected zone with a similar location and stratigraphy to the affected one. In the affected zone, the horizontal distribution of probes is made by defining equilateral triangles with a side not exceeding 1.0 m, covering the area of interest. In this configuration, the contaminant depletion rate is estimated by the difference between the heat fluxes in the affected zone in the reference. In this way, the mathematical model allows quantifying and horizontally mapping the depletion of the contaminant, which can be useful to define the area of biodegradation. In fact, this approach could be used to estimate the coverage area of a contamination by heat flux.

In addition, data on atmospheric pressure and ambient temperature, relative humidity of the air and soil, water table level in the monitored region, rainfall and solar irradiance are monitored via commercial transducers coupled to the thermodynamic source monitoring system.

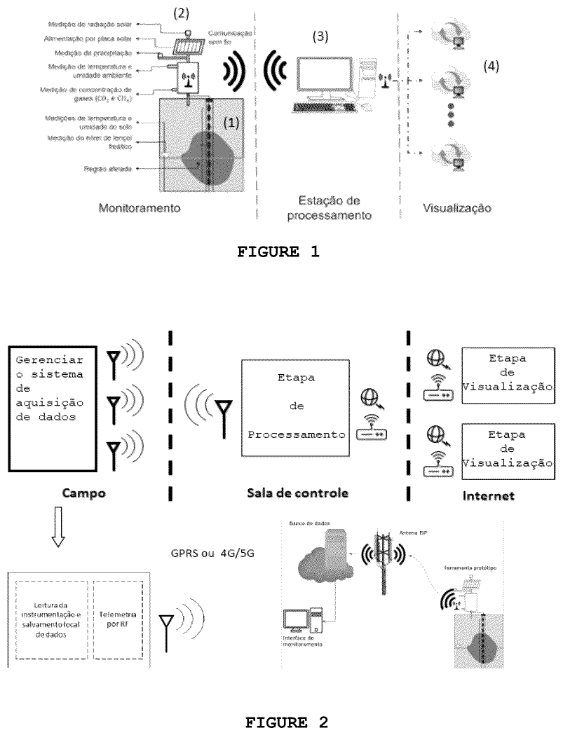

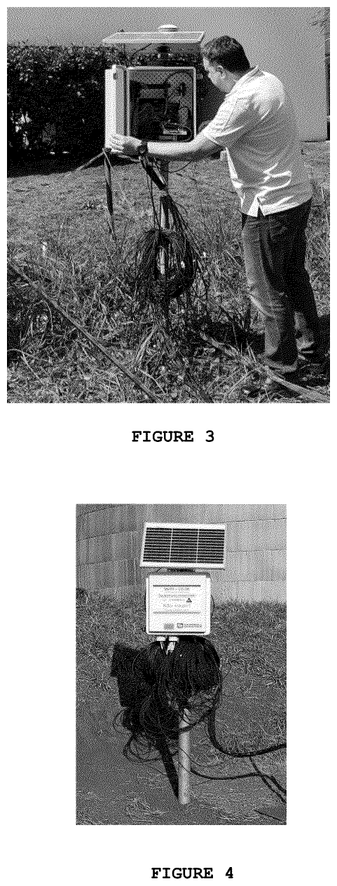

As noted, in the general scheme of the system developed for the acquisition of environmental variables of the contaminated area, with emphasis on the 1D thermal monitoring of the soil, by means of a set of heat/temperature flux transducers in FIG. 1, the station has energy autonomy through solar panels and an internal battery (2). Data transfer is carried out via radio frequency, GPRS or 3G/4G/5G.

In addition, the present invention refers to a computer-implemented method that comprises the steps of managing the data acquisition system, performing the reading of the quantities of interest, storing the measured data locally, and sending the data to a server in the cloud through a radio frequency system or mobile Internet in real time.

The method then performs the processing step which comprises receiving the transmitted data, processing the data to extract the information of interest, and making the data available for remote access to the user.

Finally, the preview step in a user interface (4) is carried out. This step involves allowing remote access to information from a computer connected to the Internet. As depicted in the general schematic illustrating the communication flow of the method in FIG. 2.

In the visualization step, the user interface (4) is a monitoring interface that allows a processing of raw data and analysis of results based on diagnostics performed on the samples collected in the field.

In a preferred embodiment of the invention, the monitoring interface obtains a main result of the estimated contaminant depletion in the source zone and the parameters that were used to model the water retention curve in the soil, as well as the values measured for the thermal conductivity and heat capacity of location samples.

In another preferred embodiment of the invention, the monitoring interface displays the installation diagram and the moment selector which enables the visualization of the instantaneous temperature profile of the soil column, the moment values of the thermodynamic variables, and the raw measurements of all the sensors of the raw data collected for that moment. In addition, in another preferred embodiment of the invention, there is a section for viewing raw data in the monitoring interface, which allows analyzing the data before it is processed to determine possible equipment failures over time.

With regard to the estimation of the mass depletion of the contaminant, the invention system acquires and processes the information to make said estimate by selecting the 1D or 2D configuration, depending on the objective of the monitoring (vertical or horizontal, i.e., in area), identifying the product (hydrocarbon) that has spilled into the soil, collecting soil samples from different depths to determine the porosity in the laboratory, the retention parameters, conductivity and heat capacity at different depths. Next, the characteristics of the probes are determined by measuring the gain and offset of each one at the time of installation, the GPS position (UTM or geographic coordinates) of the station and the vertical and horizontal positions of the probes relative to the station rod are carefully recorded, and the characteristics of the spilled substance, the soil, the probes and the assembly are inputted into a cloud database that will receive the measurements collected by the station.

Temperatures are monitored by the invention system at a monitoring station by a microcontroller using probes in a vertical or horizontal distribution, along with other environmental variables such as water table depth, rainfall, and solar irradiance. The raw information (data not processed by software) is stored locally on the station, encrypted and sent to the cloud database by radio, GPRS or 3G/4G. In the case of radio transmission, the receiver is a second station connected to a WIFI router. In the case of GPRS or 3G/4G/5G, the receiver is the ISP antenna that provides the data/telemetry plan for the station.

The treatment of the monitored raw data includes analyzing the raw data to identify drifts in the gain or offset of the thermometers and eliminate outliers caused by eventual probing failures. This is possible due to the proximity between the thermometers that make up the control of heat flux transducers and the history of measurements. Then, the temperatures and their gradients are calculated for each moment measured at each observation point using the geometry of the probes. The temperature interpolant parameters are calculated for each moment using the point temperatures and derivatives, and the internal energy of the soil (1D configuration) and heat fluxes (1D and 2D configuration) are calculated for each moment. For this, a modeling of the thermal conductivity and heat capacity of the soil is carried out as a function of the water content in all soil layers. In the case of the 1D configuration, the energy balance between the exchange of internal energy and the heat flux between consecutive moments is carried out to determine the energy produced or consumed in the control volume. The procedure is equivalent to determining the value of the source in the heat equation. In the 2D configuration, the difference between the energy flow of each probe is measured with the reference probe to determine the variation in the heat flux of the affected region. Finally, the cumulative value of energy transmitted through the plane over time for 2D configurations or energy produced per unit area for 1D configurations is calculated.

As for the results obtained, it is observed that the energy accumulated over time is an indirect measure of the amount of methane that has been oxidized in the vadose zone. In addition, the specific energy of the substance (hydrocarbon) can then be used to estimate the amount of mass that has undergone biodegradation over the course of the process in the monitored region. It is possible to use an exponential model to adjust the parameters of the decay curve and identify if there is any temporal dependence on decay rates as a result of seasonal temperature or water table level. Consequently, from the modeling of the decay rate, it is possible to make a projection of the depletion over time and define a function that calculates the time needed to reach some user-defined concentration level.

1. A thermodynamic contamination source monitoring system to determine the biodegradation rate in contaminated areas in real time, comprising:
a set of heat flux transducers distributed in the soil (1);
different plans;
a cylinder;
wherein the inside of said cylinder accommodates an installation wiring;
each flux transducer is composed of four temperature transducers arranged on the surface of a threaded cylindrical module in two planes perpendicular to the axis of said cylinder;
the system also has a station with energy autonomy (2);
wherein the system acquires the processed information to estimate the depletion of the contaminant mass through a processing station (3):
selecting 1D or 2D configuration;
identifying the product that has spilled into the soil;
collecting soil samples at different depths, in order to obtain retention, conductivity and heat capacity parameters at different depths;
determining the characteristics of the probes by measuring the gain and offset of each at the time of installation;
recording the GPS position of the station and the vertical and horizontal positions of the probes relative to the station rod; and
inserting the characteristics of the spilled substance, the soil, the probes and the assembly in a database that will receive the measurements collected by the station to the user interface (4) that allows a processing of raw data and analysis of results based on diagnostics carried out on the samples collected in the field.

2. A system, according to claim 1, wherein the heat generated by the biodegradation of petroleum hydrocarbons is monitored by said transducers.

3. A system, according to claim 1, wherein each said plane has a pair of temperature probes, some of which have measurement points at the angles 0° and 180° and others with points at the 90° and 270° angles.

4. A system, according to claim 1, wherein the diameter of said cylinder is between 3 and 10 cm, depending on the number of probes that will be attached to the rod.

5. A system, according to claim 1, wherein it is determined by a set of transducers, the temperature and its three-dimensional gradient at the center of the transducer.

6. A system, according to claim 1, wherein data on atmospheric pressure and ambient temperature, relative humidity of the air and soil, water table level in the monitored region, rainfall and solar irradiance are monitored via commercial transducers coupled to the thermodynamic source monitoring system.

7. A system, according to claim 1, wherein said station with energy autonomy (2) can comprise solar panels and an internal battery that transfers data by means of radio frequency, GPRS or 3G/4G.

8. A system, according to claim 1, wherein the quantity and distribution of said heat flux transducers and the spacing between them depends on the geometry of the source of contamination in the subsurface of the area of interest, specifically of two configurations:
a 1D configuration consisting of a set of probes distributed vertically between the soil surface and the water table; or
a 2D configuration employing a set of probes arranged horizontally at a depth of 0.5 m in the affected zone and a single probe in an unaffected zone with a similar location and stratigraphy to the affected one.

9. A computer-implemented method for handling raw data monitored by the system in real time, as defined in claim 1, wherein the method performs the steps of:
a) analyze raw data;
b) eliminate outliers caused by eventual probing failures;
c) calculate the temperatures and their gradients for each moment measured at each observation point using the geometry of probes;
d) calculate the temperature interpolant parameters for each moment using the point temperatures and derivatives;
e) calculate the internal energy of the soil and the heat fluxes for each moment;
f) set 1D configuration or 2D configuration;
g) calculate the cumulative value of energy transmitted through the plane over time for said 2D configuration or of energy produced per unit area for said 1D configuration;
h) use an exponential model to adjust the parameters of the decay curve;
i) identify if there is any temporal dependence on decay rates as a result of seasonal temperature or water table level;
j) make a projection of the depletion over time from the modeling of the decay rate; and
k) define a function that calculates the time it takes to reach some user-defined concentration level of said system.

10. A method, according to claim 9, wherein, in step (g) said energy accumulated over time comprises preferentially, an indirect measure of the amount of methane that has been oxidized in the soil vadose zone.
